(19) United States
(12) Patent Application Publication
(10) Pub. No.: US 2017/0296313 A1
(43) Pub. Date: Oct. 19, 2017

(54) Dental Scraper

(71) Applicants: Kas Kasravi, West Bloomfield, MI (US); Josephine Fermanian, Commerce Township, MI (US)

(72) Inventors: Kas Kasravi, West Bloomfield, MI (US); Josephine Fermanian, Commerce Township, MI (US)

(21) Appl. No.: 15/485,628

(22) Filed: Apr. 12, 2017

Related U.S. Application Data
(60) Provisional application No. 62/321,845, filed on Apr. 13, 2016.

(51) Int. Cl. A61C 17/00; A46B 15/00; A46B 9/04; A61B 17/24; A61C 15/04; A61C 15/02

(52) U.S. Cl. CPC A61C 17/005; A61C 15/046; A61C 15/02; A46B 9/04; A61B 17/244; A46B 15/0081; A46B 2200/1066

(57) ABSTRACT

The present invention is a consumer-oriented dental scraper, which helps the user maintain healthy teeth and gums via scraping away calculus, tartar, plaque, food particles, and other undesirable formations on teeth. The core components of the invention are a scraping head having a scraping edge and at least one scraping surface, a handle, and a finger grip, which are configured as follows: the back face of the scraping head is attached to the front end of the handle, and the finger grip is attached to the back end of the handle. The handle is optionally curved for better access to teeth. Optionally, the present invention is combined with a second scraping head, a dental pick, a flosser, a tongue scraper, or a toothbrush for greater flexibility in maintaining dental hygiene. Further, the present invention is produced in a disposable and biodegradable fashion.

### PRIORITY STATUS

This application claims the benefit of U.S. Provisional Application No. 62/321845, filed Apr. 13, 2016. The content of U.S. Provisional Application 62/321845 is incorporated herein by reference for all purposes.

### FIELD OF INVENTION

The present invention relates generally to dental care and, in particular, a consumer-oriented dental scraper.

### BACKGROUND

Effective dental care is essential for general health. Taking good care of teeth and gums can help prevent bad breath, tooth decay, and gum disease. Poor dental care is associated with adverse medical conditions, such as increased risk of heart attack, stroke, poorly controlled diabetes, and preterm labor. While regular dentist visits are important, it would be advantageous for the user to engage in regular dental care and cleaning in between dentist visits.

Much of the risk to teeth and gums is caused by accumulation of undesirable formations on teeth, such as plaque, calculus, tarter, and food particles. It would be desirable to have a dental scraper that can help the user easily scrape away undesirable formations on teeth, between teeth, and along the gum line. Furthermore, it would be desirable for such a device to be inexpensive, easy use, and optionally disposable. Still further, it would be desirable to combine the dental scraper with other dental care products for increased dental care efficacy. Despite the long standing importance of effective dental care, a complete consumer-oriented solution for cleaning the entire surface of teeth has not been introduced.

Therefore, there currently exists a need in the industry for a consumer-oriented device to help scrape away undesirable formations on teeth, between teeth, and along the gum line.

### SUMMARY OF INVENTION

The present invention advantageously fills the aforementioned deficiencies by providing a dental scraper which provides the ability for the user to clean the entire surface of teeth.

The present invention is a dental scraper, which is comprised of a handle attached to a scraping head, where the scraping head has a scraping edge. The back face of the scraping head is longitudinally attached to the front end of the handle. Thus, the user holds the handle of the dental scraper, and scrapes away undesirable formations on teeth by pressing and sliding the scraping edge on the surface of teeth, in between teeth, and along gum line.

The present invention may also have one or more of the following: a scraping edge, and at least one scraping surface. The scraping edge and the scraping surface may be curved in a concave fashion to provide optimal contact with teeth and to reach between teeth and along the gum line. The handle has an optional finger grip for ease of holding the dental scraper, where the finger grip optionally has a friction inducer, such as an abrasive surface, an indentation in the handle, or a raised tab. To maximize the strength of the handle and minimize material costs, grooves may be introduced on the surfaces of the handle. Further, the handle may be curved to allow better access to the interior surfaces of teeth.

For a more complete dental care, the dental scraper may be combined with other dental care products, such as a dental pick, a flosser, a tongue scraper, or a toothbrush. The material of the dental scraper shall be strong enough to remove undesirable formations on teeth, but not too strong to damage the tooth enamel. Furthermore, the dental scraper is optionally manufactured in a disposable fashion.

The present invention is unique when compared with other known devices and solutions because the present invention: (1) provides the ability to scrape away undesirable formations on teeth, between teeth, and along gum line; (2) provides the ability to clean the entire surface of teeth and not just the spaces between teeth; (3) is a low-cost solution and easy to use by the average user; and (4) is disposable.

The present invention is unique in that it is structurally different from other known consumer-oriented devices or solutions. More specifically, the present invention is unique due to the presence of: (1) a scraping head; (2) a scraping edge; (3) a scraping surface; (4) and low material cost compared to professional devices.

Among other things, it is an object of the present invention to provide a dental scraper that does not suffer from any of the problems or deficiencies associated with prior solutions.

It is still further an object of the present invention to improve dental care of the user. Further still, it is an object of the present invention to clean the entire surface of teeth from undesirable formations.

The present invention now will be described more fully hereinafter with reference to the accompanying drawings, which are intended to be read in conjunction with both this summary, the detailed description, and any preferred and/or particular embodiments specifically discussed or otherwise disclosed. This invention may, however, be embodied in many different forms, and should not be construed as limited to the embodiments set forth herein; rather, these embodiments are provided by way of illustration only and so that this disclosure will be thorough, complete, and will fully convey the full scope of the invention to those skilled in the art.

### BRIEF DESCRIPTION OF THE DRAWINGS

FIG. 1 (A and B) show perspective views of the dental scraper with a straight scraping edge.

FIG. 2 shows a dental scraper with a curved scraping edge and scraping surface.

FIG. 3 shows a dental scraper with a finger grip and friction inducer.

FIG. 4 shows a dental scraper with a groove.

FIG. 5 shows a dental scraper with a curved handle.

FIG. 6 shows a dental scraper with the finger grip positioned in the middle of the handle.

FIG. 7 shows a dental scraper with a second scraping head.

FIG. 8 shows a dental scraper combined with a dental pick.

FIG. 9 shows a dental scraper combined with a flosser.

FIG. 10 Shows a dental scraper combined with a tongue scraper.

FIG. 11 Shows a dental scraper combined with a toothbrush.

### DETAILED DESCRIPTION OF THE INVENTION

The present invention is directed to a dental scraper.

In its most complete version, the present invention is made up of the following components: a scraping head, a handle, a scraping edge, a scraping surface, a finger grip, a friction inducer, and a groove. The utility of the present invention as a dental care product is further enhanced by combining it with another commonly available dental care product, such as a dental pick, a flosser, a tongue scraper, or a toothbrush.

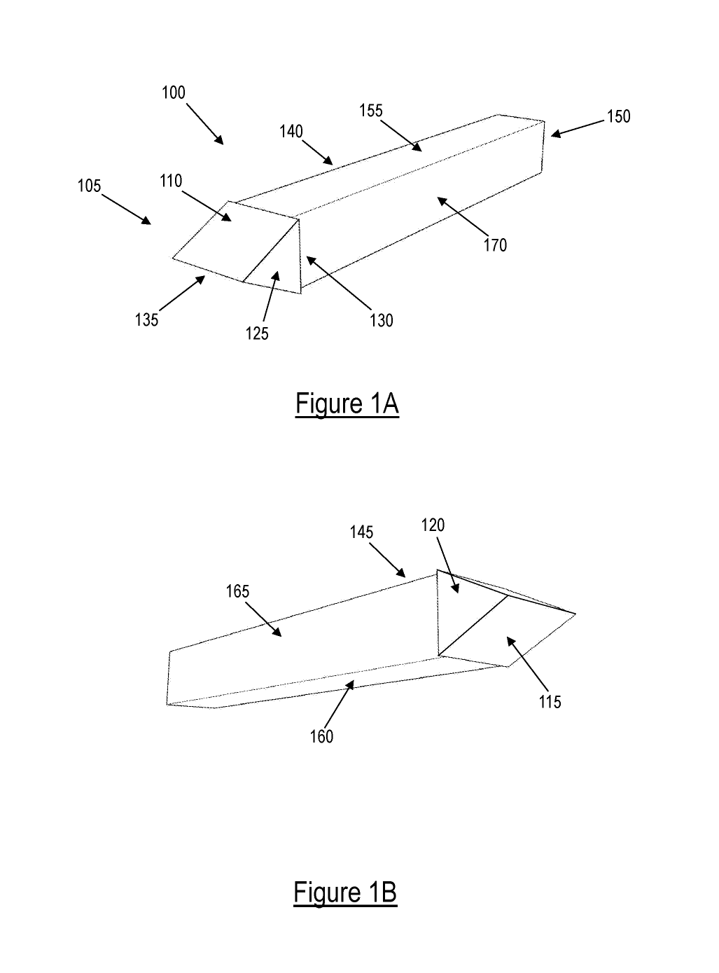

FIGS. 1A and 1B show the dental scraper 100 having a scraping head 105 and a handle 140. The scraping head 105 has a first scraping surface 110, a second scraping surface 115, a left face 120, a right face 125, and a back face 130. The first scraping face 110 and the second scraping face 115 converge away from the back face 130. A scraping edge 135 is formed via the interaction of the first scraping surface 110 and the second scraping surface 115. The handle 140 has a front end 145, a back end 150, a top side 155, a bottom side 160, a left side 165, and a right side 170. The handle 140 is attached to the scraping head 105 via the back face 130 being longitudinally attached to the front end 145. The handle 140 is held by user, who forcibly guides the scraping head 105, and, more specifically, the scraping edge 135, against the surface of teeth, between teeth, and along the gum line, to remove undesirable formations on teeth.

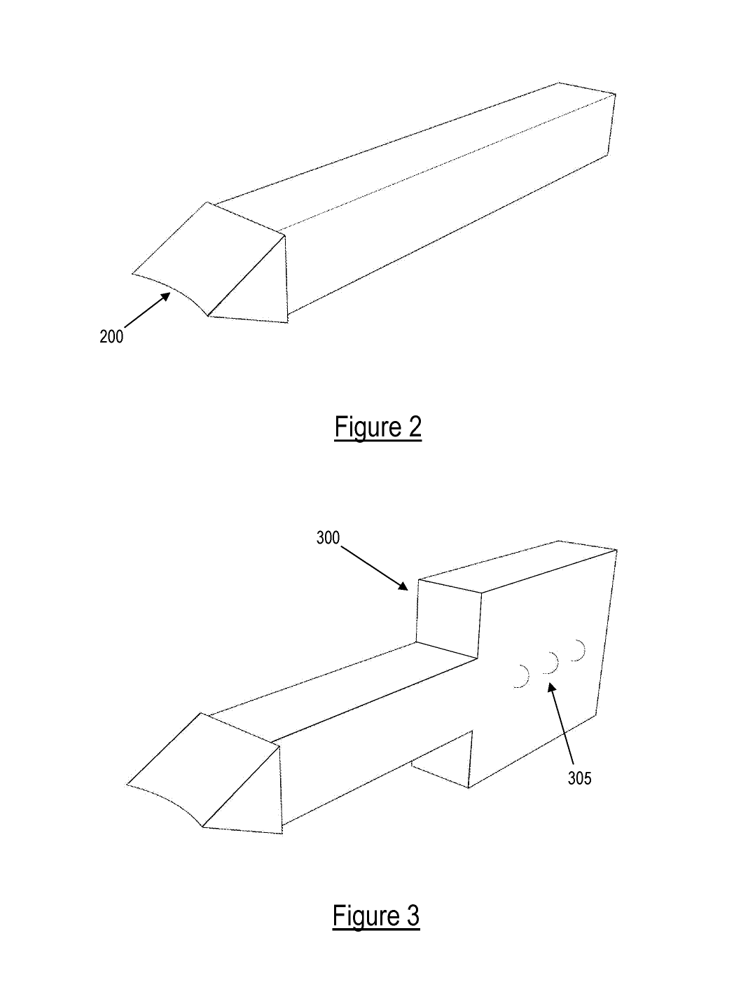

In one embodiment of the present invention, the scraping edge 135 is straight, and the first scraping surface 110 and the second scraping surface 115 are flat. With reference to FIG. 2, in the preferred embodiment of the present invention, the scraping edge 200 is curved. The aforementioned curvature is concave to provide maximum contact between the scraping edge 135 and the surface of teeth, and to reach between teeth and along the gum line.

As illustrated in FIG. 3, the preferred embodiment of the present invention, the handle 140 has a finger grip 300 positioned close to the back end 150 to provide the user with superior grasp, thus providing for a more forceful removal of undesirable formations on teeth. The finger grip 300 has a friction inducer 305 to further enhance the grasp. The friction inducer 305 assumes any commonly available grip geometry, such as an abrasive surface, an indentation, or a raised feature.

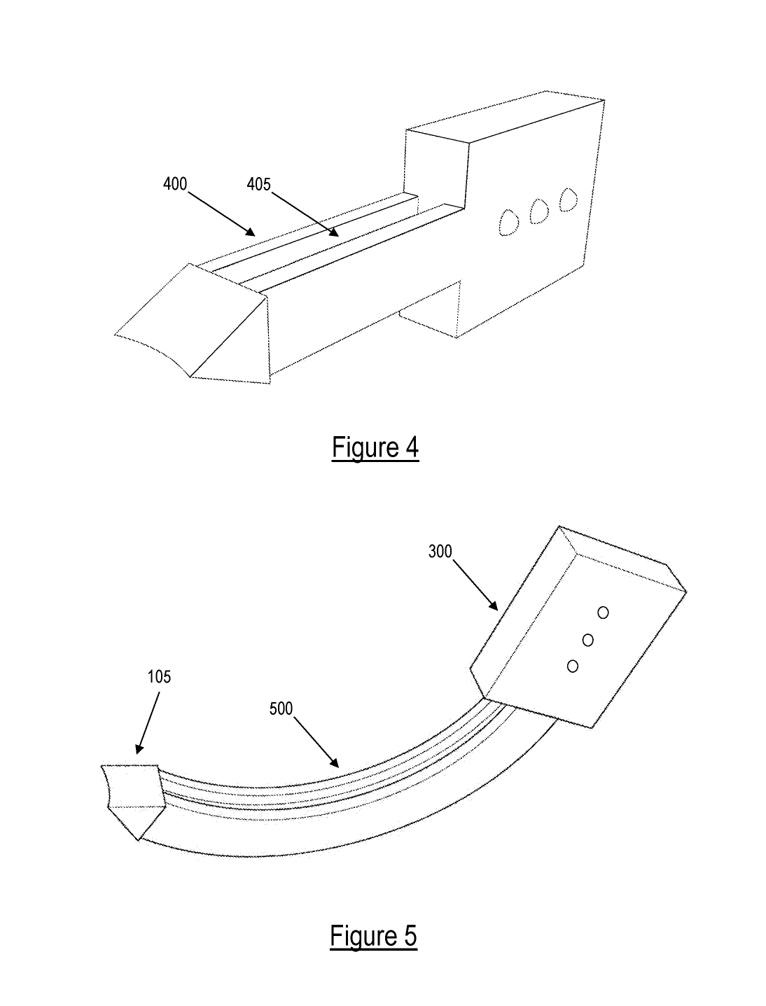

As shown in FIG. 4, the handle 400 is straight. To enhance the strength of the handle 400, reduce its weight, and reduce material requirements, a groove 405 is used on one or more sides of the handle 400.

FIG. 5 illustrates a further embodiment where handle 500 is curved between the scraping head 105 and the finger grip 300. This configuration promotes the ability to scrape the interior surfaces of teeth, as well as reaching teeth from different angles.

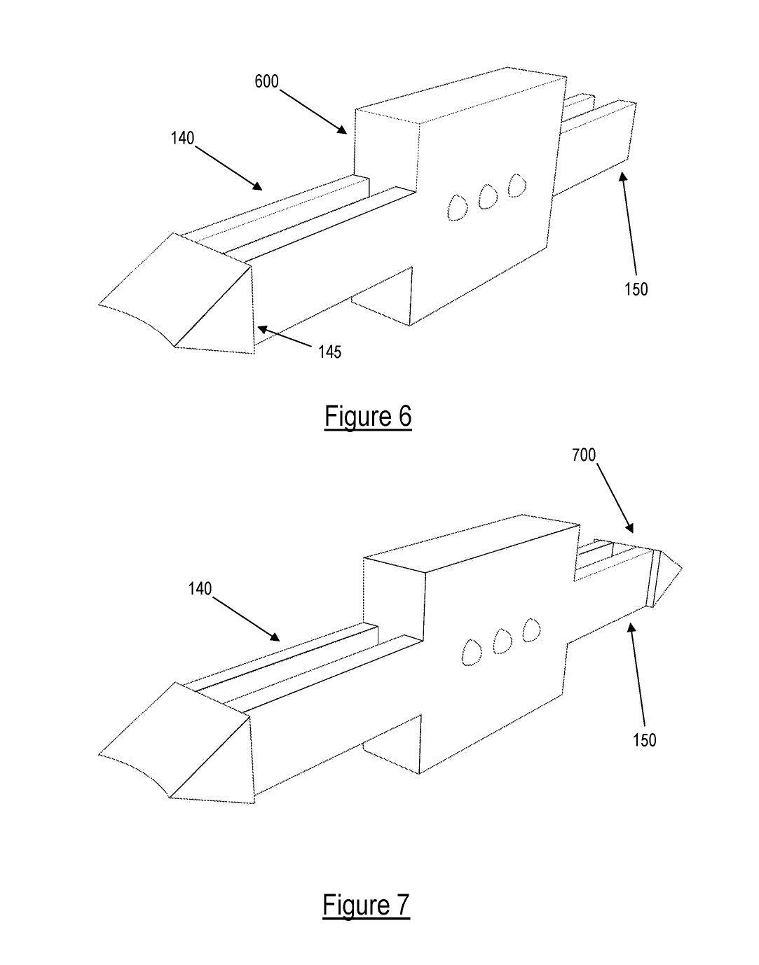

FIG. 6 shows a different embodiment of the present invention where the finger grip 600 is positioned between the front end 145 and the back end 150. FIG. 7 shows a second scraping head 700 longitudinally attached to the back end 150 of the handle 140 and pointing in the opposite direction to the first scraping head 105. Optionally, in this embodiment the scraping edges of the two scraping heads have different degrees of straightness or curvature to provide the user with an enhanced scraping experience.

To further enhance the utility of the dental scraper 100, it is optionally combined with other commonly available dental care products such as a dental pick, a flosser, a tongue scraper, or a toothbrush.

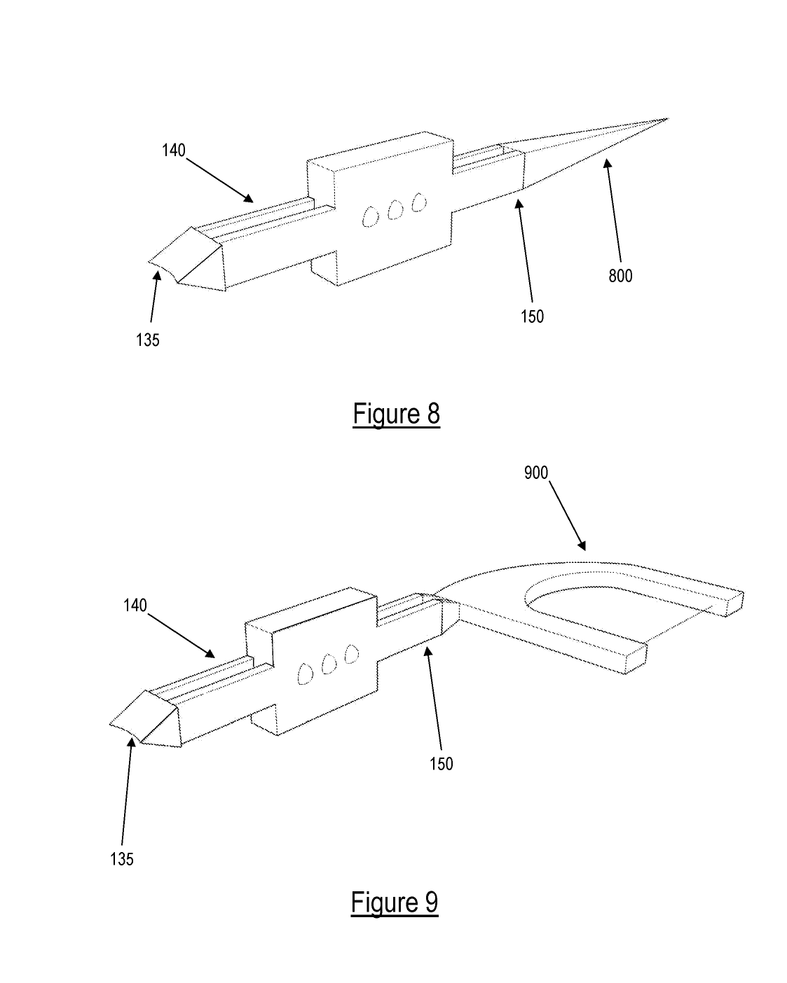

FIG. 8 illustrates an embodiment where the dental scraper 100 has a dental pick 800 longitudinally attached to the back end 150 of the handle 140. In this embodiment, the user cleans between teeth more effectively with the dental pick 800, in addition to scraping teeth with the scraping edge 135.

FIG. 9 illustrates a yet different embodiment where the dental scraper 100 has a flosser 900 longitudinally attached to the back end 150 of the handle 140. In this embodiment, the user cleans between teeth more effectively with the dental pick 800, in addition to scraping teeth with the scraping edge 135.

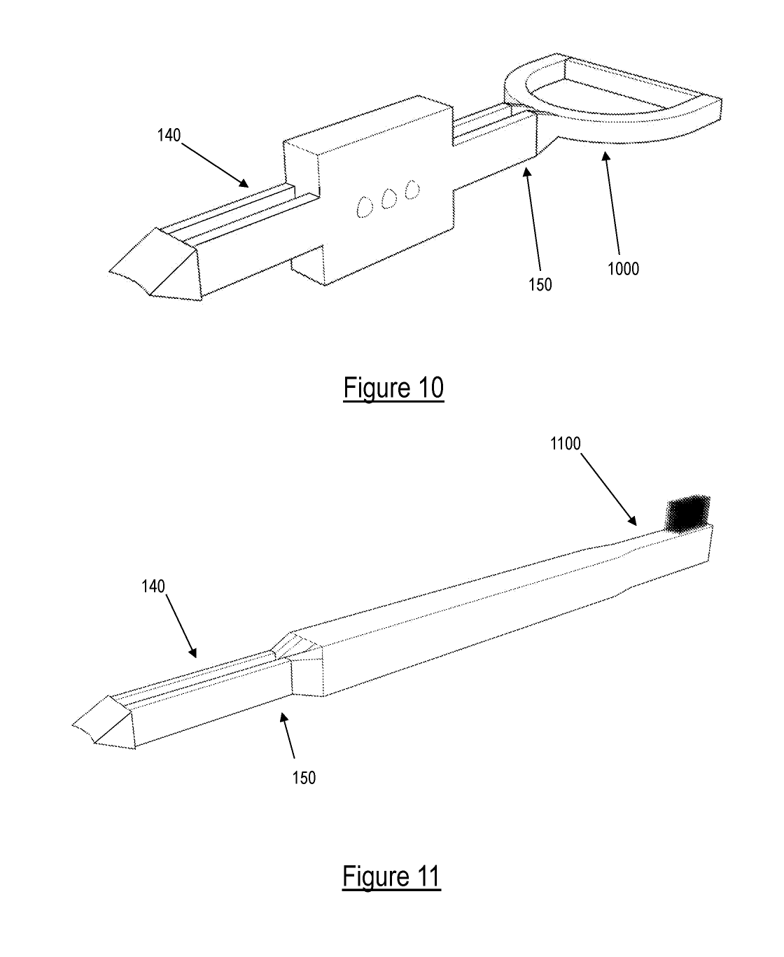

FIG. 10 shows a still different embodiment where the dental scraper 100 has a tongue scraper 1000 longitudinally attached to the back end 150 of the handle 140. In this embodiment, the user cleans the tongue with the tongue scraper 1000, in addition to scraping teeth with the scraping edge 135.

FIG. 11 illustrates another embodiment where the dental scraper 100 has a toothbrush 1100 longitudinally attached to the back end 150 of the handle 140. In this embodiment, the user cleans the teeth with the toothbrush 1100, in addition to scraping teeth with the scraping edge 135.

The material of the dental scraper 100 is selected to be soft and malleable to avoid damaging the tooth enamel, yet strong enough to scrape away undesirable formations on teeth. Further, the choice of the material is optionally influenced by recyclability, disposability, and biodegradability of the dental scraper 100. A wide range of plastics, composites, and wood material are currently available on the market for this purpose. Examples of such materials include: acrylonitrile-butadiene-styrene (ABS): polypropylene (PP): high-density polyethylene (HDPE); and Nylon. Polylactic acid (PLA) is used when recyclability and biodegradability are desired.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be, and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

We claim:

1. A dental scraper comprising:
a scraping head, having a first scraping face, a second scraping face, a left face, a right face, and a back face, where the first scraping face and the second scraping face converge opposite from the back face;
the scraping head having a scraping edge formed along the intersection of the first scraping face and the second scraping face; and
a handle, having a front end, a back end, a top side, a bottom side, a left side, and a right side, where the front end is attached to the back face of the scraping head.

2. A dental scraper according to claim 1, wherein the scraping edge is straight.

3. A dental scraper according to claim 1, wherein the scraping edge is curved in a concave manner.

4. A dental scraper according to claim 3, wherein a finger grip is attached to the back end of the handle.

5. A dental scraper according to claim 4, wherein the finger grip has a friction inducer.

6. A dental scraper according to claim 5, wherein the handle has a groove.

7. A dental scraper according to claim 6, wherein the handle is straight.

8. A dental scraper according to claim 6, wherein the handle is curved.

9. A dental scraper according to claim 3, wherein the finger grip is attached to the handle at a point between the front end and the back end of the handle.

10. A dental scraper according to claim 9, wherein the finger grip has a friction inducer.

11. A dental scraper according to claim 10, wherein the handle is straight.

12. A dental scraper according to claim 11, wherein the handle has a groove.

13. A dental scraper according to claim 12, wherein a second scraping head is attached to the back end of the handle.

14. A dental scraper according to claim 12, wherein a dental pick is attached to the back end of the handle.

15. A dental scraper according to claim 12, wherein a flosser is attached to the back end of the handle.

16. A dental scraper according to claim 12, wherein a tongue scraper is attached to the back end of the handle.

17. A dental scraper according to claim 3, wherein a toothbrush is attached to the back end of the handle.
